(19) United States
(12) Patent Application Publication
(10) Pub. No.: US 2023/0010684 A1
(43) Pub. Date: Jan. 12, 2023

(54) T-shaped Anchorable Mesh

(71) Applicant: KAOHSIUNG MEDICAL UNIVERSITY, Kaohsiung (TW)

(72) Inventor: Cheng-Yu LONG, Kaohsiung City (TW)

(21) Appl. No.: 17/857,197

(22) Filed: Jul. 5, 2022

Related U.S. Application Data
(60) Provisional application No. 63/219,844, filed on Jul. 9, 2021.

(51) Int. Cl. A61B 17/04

(52) U.S. Cl. CPC A61B 17/0401; A61B 17/0485

(57) ABSTRACT

A T-shaped anchorable mesh having a reticular structure with an intertwining mesh material, forming a tissue contact surface. The T-shaped anchorable mesh includes a long arm and a fixation part. The long arm has two lateral ends spaced from each other in an X direction and two marginal edges extending between the two lateral ends. The fixation part extends from one of the two marginal edges in a Y direction. The fixation part includes a plurality of protrusions emerging from the tissue contact surface.

### CROSS REFERENCE TO RELATED APPLICATION

The application claims the benefit of U.S. provisional application Ser. No. 63/219,844, filed on Jul. 9, 2021, and the entire contents of which are incorporated herein by reference.

### BACKGROUND OF THE INVENTION

### 1. Field of the Invention

The present invention relates to a synthetic mesh and, more particularly, to a T-shaped anchorable mesh anchored between cervix and abdominal wall fascia.

### 2. Description of the Related Art

Uterine prolapse is the displacement of the uterus beyond abdominal cavity, which means the herniation of the uterus into or beyond the vagina. The clinical symptoms uterine prolapse include bearing down sensation, frequent urination, urinary incontinence, incomplete bladder emptying and so on. There are a variety of surgical methods performed according to the level of severity in uterine prolapse. Uterine suspension surgery is the commonly used surgery in treating the uterine prolapse by fixing a synthetic mesh between cervix and the anterior longitudinal ligament of the sacrum, thereby suspending the uterus in normal position.

It is easy to slip when suturing the synthetic mesh on the cervical surface. The surgeon needs to keep holding on the synthetic mesh with one hand when performing the suture process with the other hand during the surgery. Uterine suspension surgery is usually performed laparoscopically through the insertion of laparoscopy instrument into a 5 mm laparoscopic incision wound by fixing the synthetic mesh on the cervical area, and cause more difficulties in surgical technique and inconvenience.

In view of this, there is a room of improvement of current synthetic mesh.

### SUMMARY OF THE INVENTION

To solve those above listed problems, the objective of the present invention is to provide a T-shaped anchorable mesh which has a larger frictional force with the cervical surface for better mesh attachment.

As used herein, the term “a”, “an” or “one” for describing the number of the elements and members of the present invention is used for convenience, provides the general meaning of the scope of the present invention, and should be interpreted to include one or at least one. Furthermore, unless explicitly indicated otherwise, the concept of a single component also includes the case of plural components.

As used herein, the term “coupling”, “engagement”, “assembly”, or similar terms is used to include separation of connected members without destroying the members after connection or inseparable connection of the members after connection. A person having ordinary skill in the art would be able to select according to desired demands in the material or assembly of the members to be connected.

A T-shaped anchorable mesh according to the present invention has a reticular structure with an intertwining mesh material, forming a tissue contact surface. The T-shaped anchorable mesh includes a long arm and a fixation part. The long arm includes two lateral ends spaced from each other in an X direction and two marginal edges extending between the two lateral ends. The fixation part extends from one of the two marginal edges in a Y direction. The fixation part includes a plurality of protrusions emerging from the tissue contact surface.

According to this, the T-shaped anchorable mesh of the present invention creates an uneven rough contact surface area through the plurality of protrusions on the fixation part when attaching to the tissue surface, which in turns make perfect attachment on the cervical area. Therefore, the surgeon can perform the suturing of the fixation part and abdominal oblique muscle fascia smoothly, improving the effectiveness of surgery.

In an example, each of the plurality of protrusions has a spike-like structure. Thus, the plurality of protrusions makes the tissue contact surface to be easily attached to the cervical tissue.

In an example, each of the plurality of protrusions emerges perpendicularly from the tissue contact surface. Thus, the plurality of protrusions may be slightly penetrating to the superficial area of the cervical surface, which enhances the mesh fixation to the cervical area.

In another example, each of the plurality of protrusions protrudes obliquely relative to the tissue contact surface. Thus, the T-shaped anchorable mesh has better attachment to the cervical tissue.

In a further example, the plurality of protrusions protrudes obliquely in a plurality of predetermined directions. Thus, slippage of the T-shaped anchorable mesh during mesh fixation can be avoided.

In still another example, each of the plurality of protrusions has a loop-like pattern formed by connecting two ends of a thread to form two connection points at the tissue contact surface. Thus, the fixation part can form a rough contact surface to increase the frictional force between the T-shaped anchorable mesh and the cervix for better mesh attachment.

In an example, the fixation part further includes at least one insertion pin having a pin body emerging from the tissue contact surface. Thus, the pin body of the at least one insertion pin can penetrate deeply into the cervical tissue for better mesh attachment.

In an example, the pin body includes a plurality of spikes extending towards the tissue contact surface. Thus, dislodgement of the insertion pin from the cervical area can be avoided.

In an example, the at least one insertion pin includes three insertion pins forming a triangular distribution pattern. One of the three insertion pins is fixed on an upper part of the Y direction and is relatively close to the long arm, and the other two insertion pins are relatively away from the long arm in the Y direction and are spaced from each other by a predetermined distance in the X direction. Thus, penetration and injury of the large blood vessels of cervix during fixation can be avoided.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 is a perspective view of a T-shaped anchorable mesh of a first embodiment according to the present invention.

FIG. 2 is a magnified view of a circled portion 2 of the T-shaped anchorable mesh of FIG. 1.

FIG. 3 is a side view illustrating a fixation part of the T-shaped anchorable mesh of the first embodiment according to the present invention.

FIG. 4 is a schematic diagram similar to FIG. 2, illustrating another example of protrusions.

FIG. 5 is a focal magnified view of a T-shaped anchorable mesh of a second embodiment according to the present invention.

In the various figures of the drawings, the same numerals designate the same or similar parts. Furthermore, when the terms “inner”, “outer”, “top”, “bottom”, “front”, “rear” and similar terms are used hereinafter, it should be understood that these terms have reference only to the structure shown in the drawings as it would appear to a person viewing the drawings, and are utilized only to facilitate describing the invention.

### DETAILED DESCRIPTION OF THE INVENTION

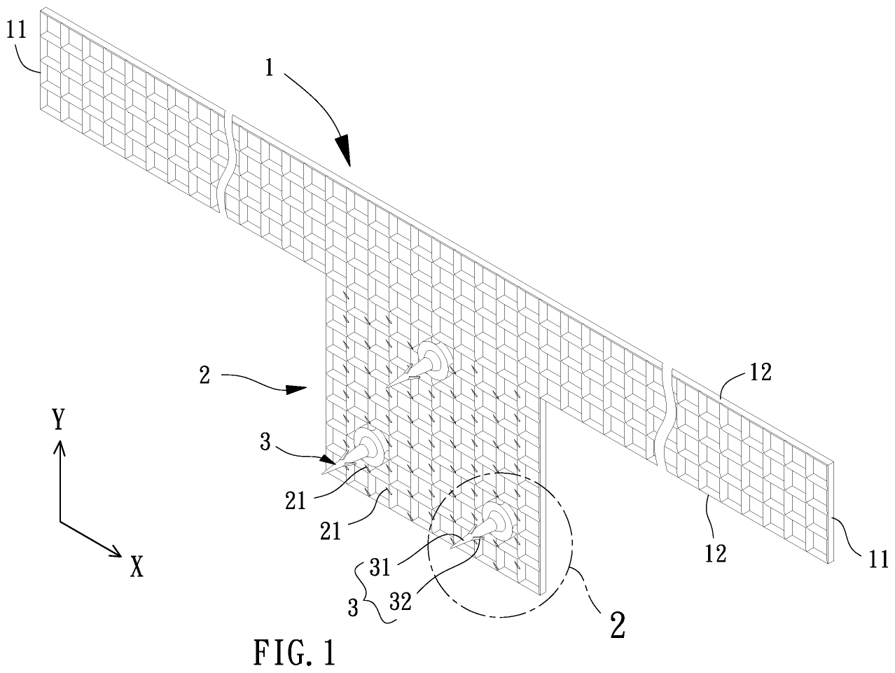

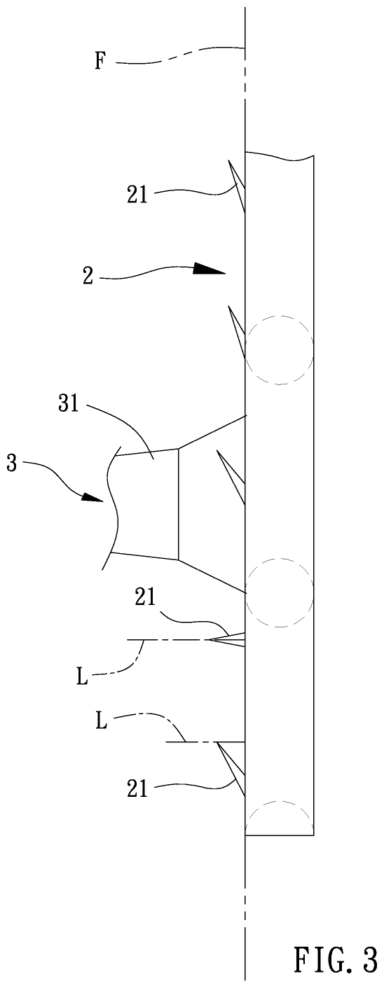

Referring to FIGS. 1 and 3, the T-shaped anchorable mesh is manufactured from polypropylene or polyvinylidene difluoride (PVDF) and will not cause human body mesh rejection reaction. The T-shaped anchorable mesh has a reticular structure made from the interlinking and intertwining of the above-mentioned mesh material. The knitting of the T-shaped anchorable mesh provides multidirectional elasticity which makes it easily attachable to the surgical site. Furthermore, the mesh can be cut into any desired shape or size such as square, rhombus or irregular shape. The T-shaped anchorable mesh includes a long arm 1 and a fixation part 2, the fixation part 2 is connected to an edge in a longitudinal direction of the long arm 1, forming the T-shape. A surface of the T-shaped anchorable mesh forms a tissue contact surface F for mesh attachment with cervical tissue.

The long arm 1 has two lateral ends 11 spaced from each other in an X direction. The two lateral ends 11 are connected to each other by two opposite marginal edges 12. The length of the long arm 1 is not limited in the present invention. The surgeon may tailor the mesh into any desired size in order to suit the surgical need.

Referring to FIGS. 1, 2 and 3, the fixing part 2 is located on one of the two marginal edges 12 of the long arm 1 and extends in a Y direction. The fixing part 2 is located approximately in a middle portion of the one of the two marginal edges 12, making the fixing part 2 spaced from each of the two lateral ends 11 by a predetermined distance. The tissue contact surface F of the fixing part 2 has a plurality of protrusions 21 for forming an uneven surface and increasing the frictional force with the cervical tissue. In this embodiment, each of the plurality of protrusions 21 has a spike-like structure enabling the tissue contact surface F to be easily attached to the cervical tissue and prevent slippage. In addition, the surgeon can suture the fixation part 2 to the cervical tissue first, followed by fixing the long arm 1 to the abdominal oblique muscle fascia in the abdominal wall when performing uterine suspension surgery. It is preferred that the plurality of protrusions 21 only exists in the tissue contact surface F of the fixation part 2 with no protrusion found on the long arm 1. Therefore, the long arm 1 has retained its flexibility during mesh tension adjustment, which makes the operation to be performed smoothly.

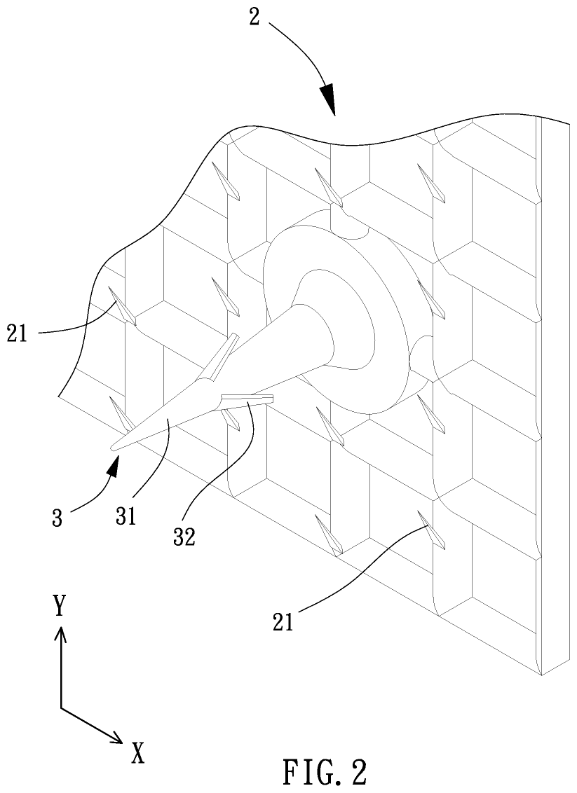

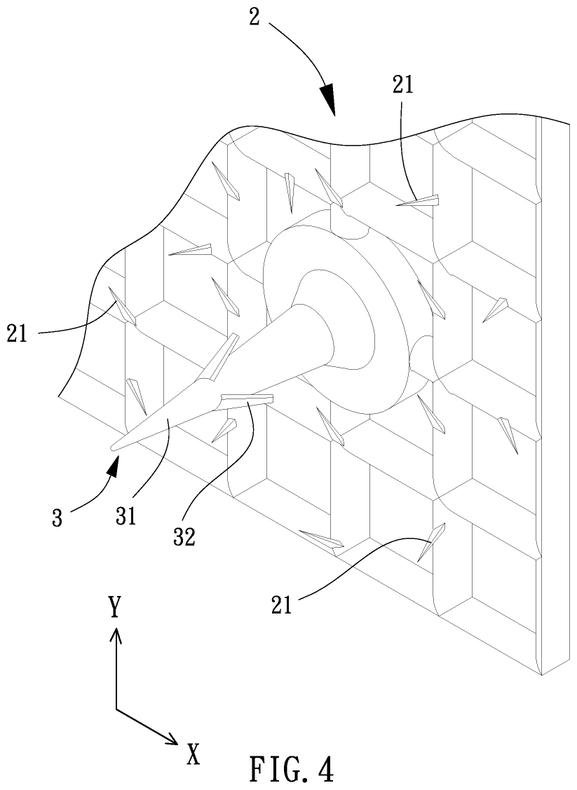

Referring to FIGS. 2 and 3, the plurality of protrusions 21 may emerge perpendicularly or non-perpendicularly from the tissue contact surface F of the T-shaped anchorable mesh. Thus, the plurality of protrusions 21 may penetrate into the superficial layer of the cervix, helps allocating the fixation part 2. For example, a tip of each of the plurality of protrusions 21 forms a reference line L perpendicular to the tissue contact surface F. An intersection point of the reference line L and the tissue contact surface F may be located within a projection area of a bottom of a respective protrusion 21, which makes the respective protrusion 21 protrude vertically from the tissue contact surface F. Alternatively, the intersection point of the reference line L and the tissue contact surface F may locate beyond the projection area, the respective protrusion 21 still can protrude obliquely to the tissue contact surface F (as shown in FIG. 3). Thus, the fixation part 2 has better fixation effect on the cervical tissue. In another embodiment, the plurality of protrusions 21 may protrude obliquely in several different directions with respect to the tissue contact surface F (as shown in FIG. 4), ensuring that the fixation part 2 does not slide easily during mesh insertion.

Referring to FIG. 1, the fixation part 2 may further include at least one insertion pin 3 which made of polypropylene or polyvinylidene difluoride (PVDF) with less mesh rejection. A pin body 31 of the at least one insertion pin 3 may be formed together with the fixation part 2 by injection molding, or the pin body 31 of the at least one insertion pin 3 may be inserted into pores of the reticular structure of the T-shaped anchorable mesh. The pin body 31 protrudes from the tissue contact surface F. For better and tight mesh fixation, the surgeons can press the pin body 31 on the fixation part 2 deeper into the cervical tissue before perform suturing between the fixation part 2 and the cervix area. Furthermore, a plurality of spikes 32 may be formed on the pin body 31 extending towards the tissue contact surface F to prevent dislodgement. In this embodiment, the fixation part 2 has three insertion pins 3 forming a triangular distribution pattern. One of the three insertion pins 3 is fixed on an upper part of the Y direction and is relatively close to the long arm 1, while the other two insertion pins 3 are relatively away from the long arm 1 in the Y direction, and are spaced from each other by a predetermined distance in the X direction. In this way, the penetration and injury of the large blood vessels of cervix during fixation are avoided.

The T-shaped anchorable mesh is utilized in uterine suspension surgery with the plurality of protrusions 21 on the fixation part 2 attached tightly to the cervical tissue in order to avoid slippage during mesh fixation. The surgeons can perform the suturing stably due to less mesh slippage and can move to subsequent surgical steps smoothly. Furthermore, the two lateral ends 11 of the long arm 1 can be pulled along the bilateral round ligaments toward 2 cm medial to Anterior superior iliac spine in abdominal wall, and finally attached to the abdominal oblique muscle fascia for ventral suspension of the uterus.

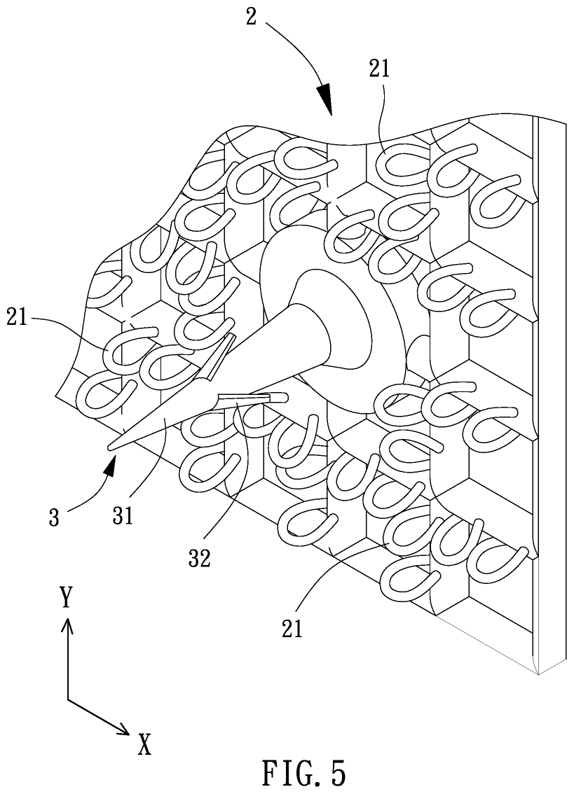

FIG. 5 shows a T-shaped anchorable mesh of a second embodiment which is substantially the same as the first embodiment. In the current embodiment, each of the plurality of protrusions 21 has a loop-like pattern. Each of the loop-like protrusions 21 is formed by connecting two ends of a thread to form two connection points at the tissue contact surface F. The plurality of protrusions 21 on the fixation part 2 are attached to the cervical area, forming a rough contact surface which helps to increase the frictional force between the T-shaped anchorable mesh and the cervix for better mesh attachment. In this embodiment, the fixation part 2 may be equipped with at least one insertion pin 3 and a plurality of spikes 32 formed on a pin body 31 of the at least one insertion pin 3 for better fixation over the cervical area.

In summary, the plurality of protrusions of the T-shaped anchorable mesh creates an uneven rough structure over the tissue contact surface which makes the fixation part perfectly adhered to the cervical area. Therefore, the surgeon can perform the suturing between the fixation part and the cervix more stably, further elevating the effectiveness of surgery.

Although the invention has been described in detail with reference to its presently preferable embodiments, it will be understood by one of ordinary skill in the art that various modifications can be made without departing from the spirit and the scope of the invention, as set forth in the appended claims.

What is claimed is:

1. A T-shaped anchorable mesh having a reticular structure with an intertwining mesh material, forming a tissue contact surface, wherein the T-shaped anchorable mesh comprises:
a long arm including two lateral ends spaced from each other in an X direction and two marginal edges extending between the two lateral ends; and
a fixation part extending from one of the two marginal edges in a Y direction, wherein the fixation part includes a plurality of protrusions emerging from the tissue contact surface.

2. The T-shaped anchorable mesh as claimed in claim 1, wherein each of the plurality of protrusions has a spike-like structure.

3. The T-shaped anchorable mesh as claimed in claim 1, wherein each of the plurality of protrusions emerges perpendicularly from the tissue contact surface.

4. The T-shaped anchorable mesh as claimed in claim 1, wherein each of the plurality of protrusions protrudes obliquely relative to the tissue contact surface.

5. The T-shaped anchorable mesh as claimed in claim 1, wherein the plurality of protrusions protrudes obliquely in a plurality of predetermined directions.

6. The T-shaped anchorable mesh as claimed in claim 1, wherein each of the plurality of protrusions has a loop-like pattern formed by connecting two ends of a thread to form two connection points at the tissue contact surface.

7. The T-shaped anchorable mesh as claimed in claim 1, wherein the fixation part further includes at least one insertion pin having a pin body emerging from the tissue contact surface.

8. The T-shaped anchorable mesh as claimed in claim 7, wherein the pin body includes a plurality of spikes extending towards the tissue contact surface.

9. The T-shaped anchorable mesh as claimed in claim 7, wherein the at least one insertion pin includes three insertion pins forming a triangular distribution pattern, wherein one of the three insertion pins is fixed on an upper part of the Y direction and is relatively close to the long arm, and wherein the other two insertion pins are relatively away from the long arm in the Y direction and are spaced from each other by a predetermined distance in the X direction.
